(19) United States
(12) Patent Application Publication
(10) Pub. No.: US 2025/0100961 A1
(43) Pub. Date: Mar. 27, 2025

(54) CHEMICAL ENTITIES FOR TREATING ACUTE MUSCULOSKELETAL PAIN, NON-SPECIFIC LOW-BACK PAIN, INFLAMMATORY ARTHRITIS RELATED DISEASES AND CONDITIONS, AND COMPOSITIONS AND METHODS THEREOF

(71) Applicant: X-Cutag Therapeutics (Suzhou) Limited, Suzhou (CN)

(72) Inventors: Hui Joyce LI, Westborough, MA (US); Changfu CHENG, Northborough, MA (US)

(73) Assignee: X-Cutag Therapeutics (Suzhou) Limited, Suzhou (CN)

(21) Appl. No.: 18/778,227

(22) Filed: Jul. 19, 2024

Related U.S. Application Data
(60) Provisional application No. 63/528,126, filed on Jul. 21, 2023.

(51) Int. Cl. C07C 271/12; A61K 31/27

(52) U.S. Cl. CPC C07C 271/12; A61K 31/27

(57) ABSTRACT

Novel deuterium- or fluorine-containing chemical compounds are provided. They are centrally acting skeletal muscle relaxants, effective for treating various types of muscle spasms, including acute musculoskeletal pain, low-back pain, inflammatory arthritis, fibromyalgia, and perioperative care for hip and knee replacements or related conditions. In addition, pharmaceutical compositions and methods for their preparation and use are described.

### TECHNICAL FIELD

The invention generally relates to therapeutics and treatment methods for muscle spasm and related diseases and conditions. Specifically, this invention provides novel chemical compounds, including CTG06-03, CTG06-04, CTG06-05 and CTG06-06, that are derived from methocarbamol, with one or more deuterium/fluorine-substitutions at strategic positions. The compounds are centrally acting skeletal muscle relaxants, effective in treating various types of muscle spasms including acute pain of musculoskeletal origin, low-back pain, inflammatory arthritis, fibromyalgia, perioperative care of hip and knee replacements or related diseases and conditions. The invention further relates to pharmaceutical compositions and methods of preparation and use thereof.

### BACKGROUND OF THE INVENTION

Muscle spasms are common occurrence in our daily lives. They are often accompanied by pain, discomfort, and a sensation of tightness or cramping in the affected muscles. It can affect various parts of the body, including legs, back, neck, arm and abdomen, making it challenging to engage in normal physical activities or perform certain activities or tasks.

Methocarbamol has been widely used in clinical practice as a muscle relaxant for muscle spasms since 1957 (LiverTox: Clinical and Research Information on Drug-Induced Liver Injury,” in Methocarbamol, Bethesda (MD), Diseases, National Institute of Diabetes and Digestive and Kidney, 2017). Classified as a centrally acting skeletal muscle relaxant (Elenbass, 1980), methocarbamol is primarily used as anti-spasmodic agent to alleviate muscle spasms and associated pain. This includes a range of conditions involving musculoskeletal injuries, strains and sprains, as well as managing conditions such as low back pain, neck pain, inflammatory arthritis, fibromyalgia, rib fractures, perioperative hip and knee replacement and abdominal muscle cramps in cirrhosis patients (Richards B L, et al., 2012, Beebe F A, et. al 2015, Patanwala A E, et. al 2017, Looke T D, et al., 2013, Abd-Elsalam S, et al., 2019, Foster N E, et al., 2018).

Even though the exact mechanism of action of methocarbamol and the relationship between musculoskeletal pain and muscle spasm are not fully elucidated, it is believed that methocarbamol exerts its effects by depressing the central nervous system and inhibiting the transmission of nerve impulses or pain signals to the brain (Truitt E B, et al., 1957). By modulating these signals, methocarbamol helps to relax muscles and relieve muscle spasms, contributing to improved comfort and mobility for patients. Methocarbamol has no direct effect on skeletal muscle contractility or motor nerve fiber (O'Doherty D S, 1958).

Orally ingested methocarbamol typically begins to take effect within 30 minutes after administration and reaches peak plasma concentration at two hours (Elenbass, 1980). The half-life of methocarbamol ranges from 1 to 2 hours. It is metabolized in the liver and excreted through the urine as an inactive metabolite. No liver toxicity is observed.

As with any medication, methocarbamol may have potential side effects, including drowsiness, dizziness, blurred vision, headache, and upset stomach. Consequently, individuals taking methocarbamol should exercise caution when engaging in activities that require mental alertness, such as driving or operating machinery, as the medication may induce drowsiness.

Methocarbamol has over three million prescriptions each year in the United States. It was found to be more effective for acute musculoskeletal pain (Chou R et al., 2004). In addition to its established clinical usage, there is potential to extend the use for other symptoms including low back pain, neck pain, inflammatory arthritis, fibromyalgia, and rib fractures. However, considering the potential side effects associated with methocarbamol, there is a need for an enhanced formulation that offers increased potency and a longer half-life. Such advancements would contribute to improved patient outcomes in the use of methocarbamol.

### BRIEF SUMMARY OF THE INVENTION

This invention generally relates to therapeutics and treatment methods for spasms that are common occurrence in our daily lives. The new chemical entities arise from the modification of methocarbamol, a muscle relaxant for treating spasms since 1957. This invention provides novel chemical compounds, including CTG06-03, CTG06-04, CTG06-05 and CTG06-06, that are derived from methocarbamol, with one or more deuterium/fluorine-substitutions at strategic positions. The compounds are centrally acting skeletal muscle relaxants and useful for treating various types of muscle spasms including acute pain of musculoskeletal origin, low-back pain, inflammatory arthritis, fibromyalgia, perioperative care of hip and knee replacements or related diseases and conditions. The invention further relates to pharmaceutical compositions and methods of preparation and use thereof. Specifically, the new chemical entities are fluorine/deuterium substituted compounds of methocarbamol.

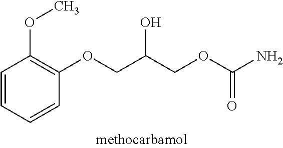 [SMILES: COc1ccccc1OCC(O)COC(N)=O]

The compounds disclosed herein are modified versions of methocarbamol, wherein one or more hydrogen atoms are substituted with a deuterium/fluorine atom at strategic locations. The inventive compounds of the present invention are represented by the following Formula (I), wherein each of R¹, R² and R³ is independently selected from H, D, or F, wherein R³ can also be OH, and at least one of R¹, R², and R³ is D or F.

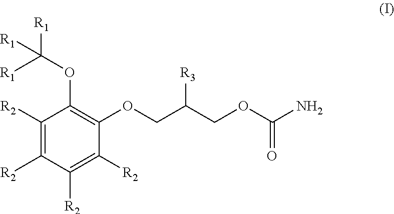

In another aspect, the invention generally relates to a pharmaceutical composition comprising a compound of the Formula (I) or a pharmaceutically acceptable salt or ester thereof, that is a centrally acting skeletal muscle relaxant and useful for treating various types of muscle spasms including acute pain of musculoskeletal origin, low-back pain, inflammatory arthritis, fibromyalgia, perioperative care of hip and knee replacements or related diseases and conditions.

In another aspect, the invention generally relates to a dosage form comprising a compound of the Formula (I), wherein the dosage form is suitable for administrating to a subject, animal or human, that suffers from various types of muscle spasms including acute pain of musculoskeletal origin, low-back pain, inflammatory arthritis, fibromyalgia, perioperative care of hip and knee replacements or related diseases and conditions.

In another aspect, the invention generally relates to a method of producing or using a compound of the Formula (I).

The foregoing has outlined rather broadly the features and technical advantages of the invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description. It is to be expressly understood, however, that any description, figure, example, etc. is provided for the purpose of illustration and description only and is by no means intended to define the limits of the invention.

### BRIEF DESCRIPTION OF THE DRAWINGS

FIG. 1. Comparison of the remaining percentage of CTG06-05 (dashed line) to that of methocarbamol (solid line) after incubating in human microsomes. Samples were analyzed by LC-MS/MS.

FIG. 2. Comparison of the concentrations of CTG06-05 (dashed line) and methocarbamol (solid line) in mouse plasma after 1/1 co-administration, showing a lower C₀ and a longer T_{1/2}.

FIG. 3. Concentration ratio of CTG06-05 and methocarbamol in mouse plasma after 1/1 co-administration, indicating a differentiation in drug distribution volumes.

FIG. 4. LC-MS/MS analysis of rat urine samples collected after separate administration of methocarbamol (top panel) and CTG06-06 (lower panel), showing that replacing the OH by F eliminates the drug conjugation reaction in rats.

### DETAILED DESCRIPTION OF THE INVENTION

### I. Definitions

Unless otherwise noted, technical terms are used according to conventional usage.

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

The term “subject” refers to any animal (e.g., a mammal), including non-human primates, rodents, and the like, which is to be the recipient of a particular treatment. Typically, the terms “subject” and “patient” are used interchangeably herein in reference to a human subject.

The term “effective” as used in connection with an amount of an active agent, refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the patient.

The terms “treating, reducing, or preventing” a condition refer to ameliorating such a condition before or after it has occurred. As compared with an equivalent untreated control, such reduction or degree of prevention is at least 5%, 10%, 20%, 40%, 50%, 60%, 80%, 90%, 95%, or 100% as measured by any standard technique.

The term “pharmaceutically acceptable excipient, carrier, or diluent”, as used herein, refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; dextrin and cyclodextrin (alpha, beta and gamma); powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

### II. The Present Invention

This invention provides novel chemical compounds, including CTG06-03, CTG06-04, CTG06-05 and CTG06-06, that are derived from methocarbamol, with one or more deuterium/fluorine-substitutions at strategic positions. The compounds are centrally acting skeletal muscle relaxants and useful for treating various types of muscle spasms including acute pain of musculoskeletal origin, low-back pain, inflammatory arthritis, fibromyalgia, perioperative care of hip and knee replacements or related diseases and conditions. The invention further relates to pharmaceutical compositions and methods of preparation and use thereof.

In the present invention, all compounds disclosed herein have the general structure of Formula (I), wherein each of R¹, R² and R³ is independently selected from H, D, or F, wherein R³ can also be OH, and at least one of R¹, R², and R³ is D or F.

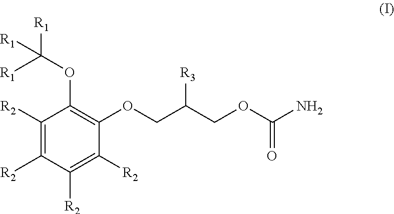

In another aspect, the invention generally relates to a pharmaceutical composition comprising a compound of the Formula (I) or a pharmaceutically acceptable salt or ester thereof, that is a centrally acting skeletal muscle relaxant and useful for treating various types of muscle spasms including acute pain of musculoskeletal origin, low-back pain, inflammatory arthritis, fibromyalgia, perioperative care of hip and knee replacements or related diseases and conditions.

In another aspect, the invention generally relates to a dosage form comprising a compound of the Formula (I), wherein the dosage form is suitable for administrating to a subject, animal or human, that suffers from various types of muscle spasms including acute pain of musculoskeletal origin, low-back pain, inflammatory arthritis, fibromyalgia, perioperative care of hip and knee replacements or related diseases and conditions.

In another aspect, the invention generally relates to a method of producing or using a compound of the Formula (I).

In certain embodiments, each of R¹ in the compound is D, each of R² is H, and R³ is OH, and is represented by the structural Formula (II) (also referred to as “CTG06-03”).

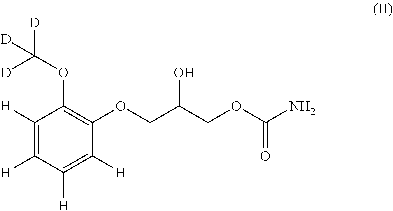 [SMILES: [2H]C([2H])([2H])Oc1ccccc1OCC(O)COC(N)=O]

In another aspect, the invention generally relates to a pharmaceutical composition comprising a compound of the Formula (II) or a pharmaceutically acceptable salt or ester thereof, that is a centrally acting skeletal muscle relaxant and useful for treating various types of muscle spasms including acute pain of musculoskeletal origin, low-back pain, inflammatory arthritis, fibromyalgia, perioperative care of hip and knee replacements or related diseases and conditions.

In another aspect, the invention generally relates to a dosage form comprising a compound of the Formula (II), wherein the dosage form is suitable for administrating to a subject, animal or human, that suffers from various types of muscle spasms including acute pain of musculoskeletal origin, low-back pain, inflammatory arthritis, fibromyalgia, perioperative care of hip and knee replacements or related diseases and conditions.

In another aspect, the invention generally relates to a method of producing or using a compound of the Formula (II).

In certain embodiments, each of R¹ in the compound is F, each of R² is H, and R³ is OH, and is represented by the structural Formula (III) (also referred to as “CTG06-04”).

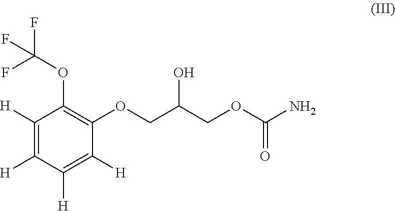 [SMILES: NC(=O)OCC(O)COc1ccccc1OC(F)(F)F]

In another aspect, the invention generally relates to a pharmaceutical composition comprising a compound of the Formula (III) or a pharmaceutically acceptable salt or ester thereof, that is a centrally acting skeletal muscle relaxant and useful for treating various types of muscle spasms including acute pain of musculoskeletal origin, low-back pain, inflammatory arthritis, fibromyalgia, perioperative care of hip and knee replacements or related diseases and conditions.

In another aspect, the invention generally relates to a dosage form comprising a compound of the Formula (III), wherein the dosage form is suitable for administrating to a subject, animal or human, that suffers from various types of muscle spasms including acute pain of musculoskeletal origin, low-back pain, inflammatory arthritis, fibromyalgia, perioperative care of hip and knee replacements or related diseases and conditions.

In another aspect, the invention generally relates to a method of producing or using a compound of the Formula (III).

In certain embodiments, each of R¹ and R² is D, and R³ is OH, and is represented by the structural formula (IV) (also referred to as “CTG06-05”).

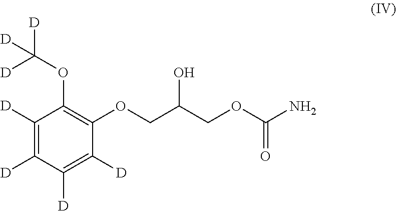 [SMILES: [2H]c1c([2H])c([2H])c(OC([2H])([2H])[2H])c(OCC(O)COC(N)=O)c1[2H]]

In another aspect, the invention generally relates to a pharmaceutical composition comprising a compound of the Formula (IV) or a pharmaceutically acceptable salt or ester thereof, that is a centrally acting skeletal muscle relaxant and useful for treating various types of muscle spasms including acute pain of musculoskeletal origin, low-back pain, inflammatory arthritis, fibromyalgia, perioperative care of hip and knee replacements or related diseases and conditions.

In another aspect, the invention generally relates to a dosage form comprising a compound of the Formula (IV), wherein the dosage form is suitable for administrating to a subject, animal or human, that suffers from various types of muscle spasms including acute pain of musculoskeletal origin, low-back pain, inflammatory arthritis, fibromyalgia, perioperative care of hip and knee replacements or related diseases and conditions.

In another aspect, the invention generally relates to a method of producing or using a compound of the Formula (IV).

In certain embodiments, each of R¹ and R² is H, and R³ is F, and is represented by the structural formula (V) (also referred to as “CTG06-06”).

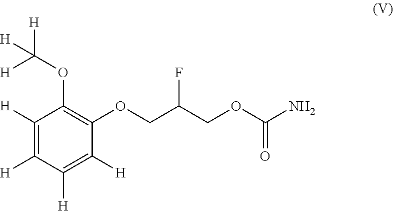 [SMILES: COc1ccccc1OCC(F)COC(N)=O]

In another aspect, the invention generally relates to a pharmaceutical composition comprising a compound of the Formula (V) or a pharmaceutically acceptable salt or ester thereof, that is a centrally acting skeletal muscle relaxant and useful for treating various types of muscle spasms including acute pain of musculoskeletal origin, low-back pain, inflammatory arthritis, fibromyalgia, perioperative care of hip and knee replacements or related diseases and conditions.

In another aspect, the invention generally relates to a dosage form comprising a compound of the Formula (V), wherein the dosage form is suitable for administrating to a subject, animal or human, that suffers from various types of muscle spasms including acute pain of musculoskeletal origin, low-back pain, inflammatory arthritis, fibromyalgia, perioperative care of hip and knee replacements or related diseases and conditions.

In another aspect, the invention generally relates to a method of producing or using a compound of the Formula (V).

Compounds in the invention were synthesized and in vitro and in vivo experiments were carried out to characterize these compounds. Experimental results showed that the compounds provided herein differentiated from methocarbamol in the following areas.

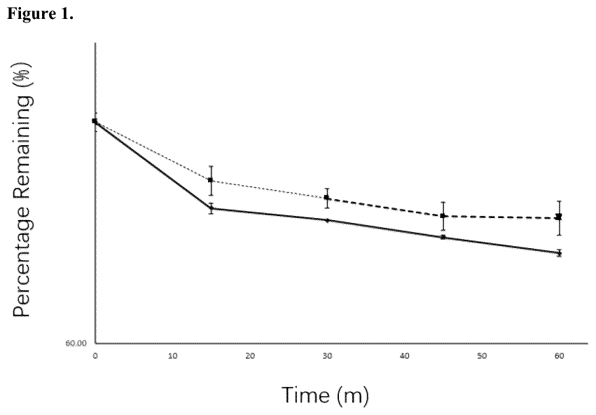

Differentiation in drug elimination half-lives. Methocarbamol and CTG06-05 were co-incubated in rat liver microsomes (1/1) and the comparison results were shown in FIG. 1. Clearly, CTG06-05 exhibits a longer drug elimination half-life compared to methocarbamol. The elimination half-life (T_{1/2}) is the time it takes for the elimination processes to reduce the concentration level or the amount of drug in the system by 50%. Elimination half-life is a composite pharmacokinetic parameter determined by both clearance and volume of distribution (V_{d}). Elimination half-life is increased by an increase in volume of distribution or a decrease in clearance, and vice versa.

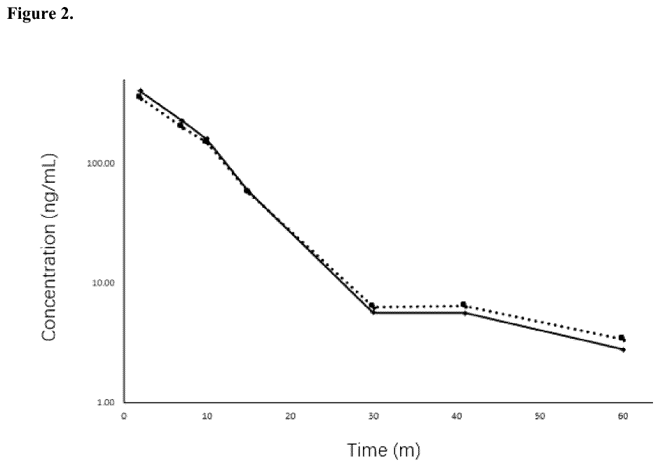

Differentiation in drug toxicity profiles. Methocarbamol and CTG06-05 were co-administered in rats and plasma samples were collected at various timepoints. The time-concentration curves of the two compounds were shown in FIG. 2. Interestingly, the initial concentration of CTG06-05 in rat plasma was lower than that of methocarbamol. However, due to its longer elimination half-life, the concentration of CTG06-05 eventually exceeded that of methocarbamol in rat plasma. This indicates a distinct toxicity profile for CTG06-05 compared to methocarbamol, particularly when the toxicity is driven by Cₘₐₓ. Cₘₐₓ is the maximum (or peak) plasma concentration that a drug achieves in a specified compartment or test area of the body after the drug has been administered.

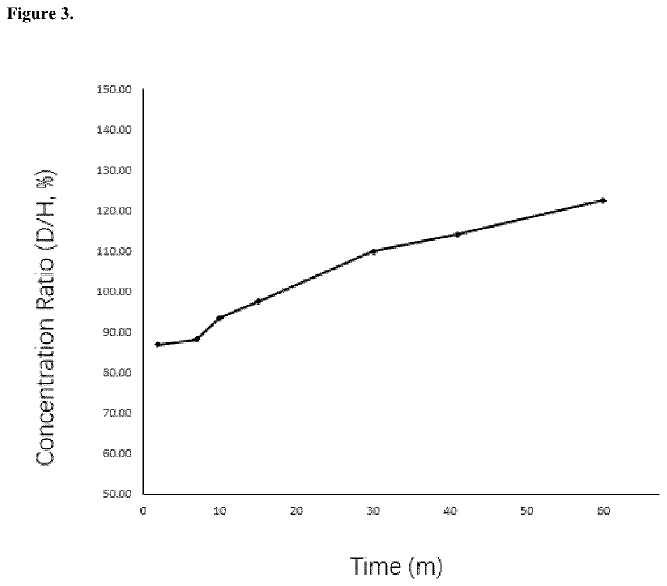

Differentiation in drug distribution volumes. Concentration ratios of CTG06-05 to methocarbamol in rat plasma samples from the aforementioned experiment were determined. FIG. 3 illustrated that the ratio of CTG06-05 to methocarbamol increased with time, indicating a differentiation in volume distribution between the two compounds. Drug distribution volume (V_{d}) is defined as the total amount of drug in the body divided by its concentration in plasma. V_{d} reflects the degree to which the drug is present in extravascular tissues rather than in the plasma. Therefore, when the total amount of drug is the same, for example, 1:1 co-administration, drug plasma concentration is a direct parameter in V_{d} comparison. In other words, the higher the concentration, the lower the V_{d}. Thus, CTG06-05 has a lower V_{d} value than methocarbamol, indicating a lower dose of CTG06-05 is required in future clinical usage.

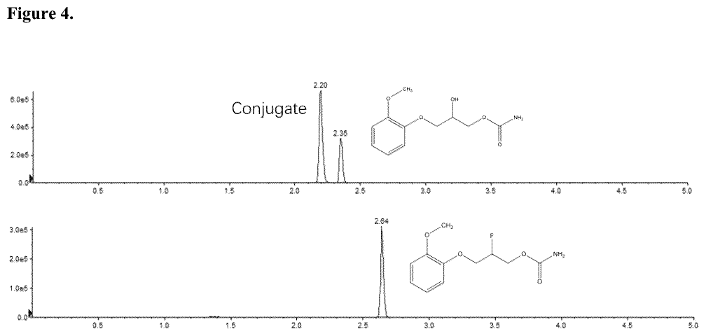

Differentiation in drug conjugation reactions. CTG06-06 and methocarbamol were co-administered to rats, followed by collecting rat urine samples for analysis using LC-MS/MS to detect the parent drug and its conjugates. As shown in FIG. 4, methocarbamol and its conjugates were detected (top panel) whereas no conjugates of CTG06-06 were detected (bottom panel). Conjugation is a phase II metabolism reaction in which an acid, for example, glucuronic acid, acts as a conjugating agent to link to a phase I metabolite covalently. The conjugate so formed would be excreted to urine. To most drugs, this is a necessary detoxicating process. As for methocarbamol, this could be a disadvantage because the acid directly forms a conjugate with the drug itself, not its metabolite, and thus, to reduce drug plasma concentration level immediately after administration. This is due to the presence of an OH group in the chemical structure of methocarbamol, which readily allows it to form conjugates with an acid. To inhibit this pathway, CTG06-06 was designed with the OH group replaced by a fluorine (F). This modification is anticipated to slow the drug elimination rate while maintaining a consistently high drug plasma concentration.

### EXAMPLES

### Example 1: Incubation of Methocarbamol and the Compounds Described in the Invention in Animal and Human Microsomes, Followed by In Vitro Sample Analysis by LC-MS/MS

Reagents: Dimethyl sulfoxide (DMSO) and acetonitrile (ACN) were purchased from VWR (Radnor, PA). 10×PBS buffer pH 7.4 was purchased from Thermo-Fischer Scientific (Waltham, MA). Ethylenediaminetetraacetic acid (EDTA) was purchased from Sigma-Aldrich (St. Louis, MO). Type I water was produced using a MilliporeSigma reverse osmosis filtration system (Burlington, MA). Dextromethorphan, methocarbamol, CTG06-03, CTG06-04, CTG06-05 and CTG06-06 were provided by Ascendex Scientific (Levittown, PA). Sodium hydroxide (NaOH) was purchased from Fisher Scientific (Hampton, NH). Human, beagle, rat and mouse microsomes, as well as NADPH regenerating system Solutions A and B, were purchased from Corning (Corning-Gentest, Tewksvury, MA).

Preparation of Primary Stock Solution and Working Solution: Each of the compounds used in this disclosure (methocarbamol, CTG06-03, CTG06-04, CTG06-05 and CTG06-06) was weighed out in powder form and dissolved in DMSO to create primary stock solutions at concentrations of 10 mM. An aliquot of each primary solution was subsequently diluted to 100 μM in sample dilution buffer. The 100 μM solutions were then further diluted to 100 nM. Dextromethorphan was weighed out in powder form and dissolved in DMSO to create a primary stock solution with a concentration of 10 mM. The 10 mM stock was serially diluted in sample dilution buffer to create a working solution with a concentration of 100 nM. Each solution, upon dilution, was vortexed for at least two minutes to ensure homogeneity.

Preparation of Sample Dilution Buffer: To prepare 0.5 M EDTA (pH8.0) solution, di-sodium EDTA was dissolved in Type 1 water and pH was adjusted to 8.0 using sodium hydroxide. 1×PBS buffer (pH7.4) was prepared by diluting 10×PBS buffer (pH 7.4) with Type 1 water. Sample dilution buffer was made by adding 1 mL of 0.5 M EDTA (pH8.0) solution to 99 mL of 1×PBS buffer (pH 7.4).

Preparation of Microsome Solution: Human pooled liver microsomes were purchased from Corning-Gentest (Tewksbury, MA) at a concentration of 20 mg protein/mL. They were thawed slowly on ice and diluted to 5 mg/mL by combining three parts of 1×PBS with one part of 20 mg/mL microsome immediately before each experiment.

Preparation of Reaction Mixture: The microsome reaction mixtures were prepared as follows: for a total volume of 285 μL of reaction mixture, 274.5 μL of 1×PBS, 3 μL of the test compound, and 7.5 μL of 5 mg/mL microsomes were added. As a negative control, microsome-free samples were prepared by substituting the microsome solution in the reaction mixture with an equal volume of 1×Phosphate Buffer Solution (pH 7.4). The reaction mixture was prepared in triplicates. In addition, dextromethorphan was used in this assay as a positive control.

Incubation and Processing of Reaction: The reaction mixture was initiated by adding 10 μL of NADPH and incubated in a water bath at 37° C. At specified time intervals (0, 15, 30, 45, 60 and 120 minutes), a 50 μL aliquot of reaction mixture was transferred from the incubation tube to a clean tube containing 250 μL of ice-cold acetonitrile to stop the reaction. The terminated samples were mixed thoroughly by vortexing and then centrifuged at 13,000 RPM for five minutes. A portion of the supernatant (˜100 μL) was transferred to an autosampler vial and subjected to liquid chromatography-mass spectrometry (LC-MS) analysis.

LC-MS/MS analysis: Reversed-phase liquid chromatographic separation was carried out using ACE 3 C18 column (50×2.1 mm id, 3 μm). Mobile phase A consisted of water with 0.1% formic acid, and mobile phase B consisted of acetonitrile with 0.1% formic acid, with a total flow rate of 0.3 mL/min. The gradient elution used was as follows: starting at 10% B at 0 min, increasing to 90% B at 2 min, maintaining 90% B till 2.5 min, and returning to 10% B at 2.6 min. Compounds were monitored in positive mode over a five-minute acquisition period using a triple quadrupole mass spectrometer. The sample runs and data processing were conducted by Analyst version 1.6.2.

Results from co-incubation studies: The compounds described in this invention demonstrated greater stability compared to methocarbamol when co-incubated in microsomes at a 1:1 ratio. The remaining percentage of CTG06-05 (dashed line) and methocarbamol (solid line) after incubating in human microsomes were compared, as shown in FIG. 1.

### Example 2: Co-Administration of CTG06-05 and Methocarbamol (1:1) to Mice and LC-MS/MS Analysis of Mouse Plasma Samples

Mouse plasma samples were collected at intervals of 2, 7, 10, 15, 30, 45, and 60 minutes after an IV dose of CTG06-05 and methocarbamol at a 1:1 ratio (0.5 mg/kg each). Plasma samples were extracted via protein precipitation. The supernatant at each time point was transferred to a clean vial and analyzed by LC-MS/MS as described in Example 1. FIG. 2 showed the concentration ratios of CTG06-05 to methocarbamol at various time points and a differentiation in drug distribution volumes was shown in FIG. 3.

### Example 3: Co-Administration of CTG06-06 and Methocarbamol (1:1) to Rats and LC-MS/MS Analysis of Rat Urine Samples

Rat urine samples were collected after an oral dose of CTG06-06 and methocarbamol at 1:1 ratio (0.5 mg/kg each). The samples were centrifuged and analyzed by LC-MS/MS described in Example 1, as shown in FIG. 4.

### Example 4: Synthesis of CTG06-05. The Routes for the Synthesis of CTG06-03, CTG06-04 and CTG06-06 are Similar to Those of CTG06-05

### CTG06-05 Synthetic Procedure

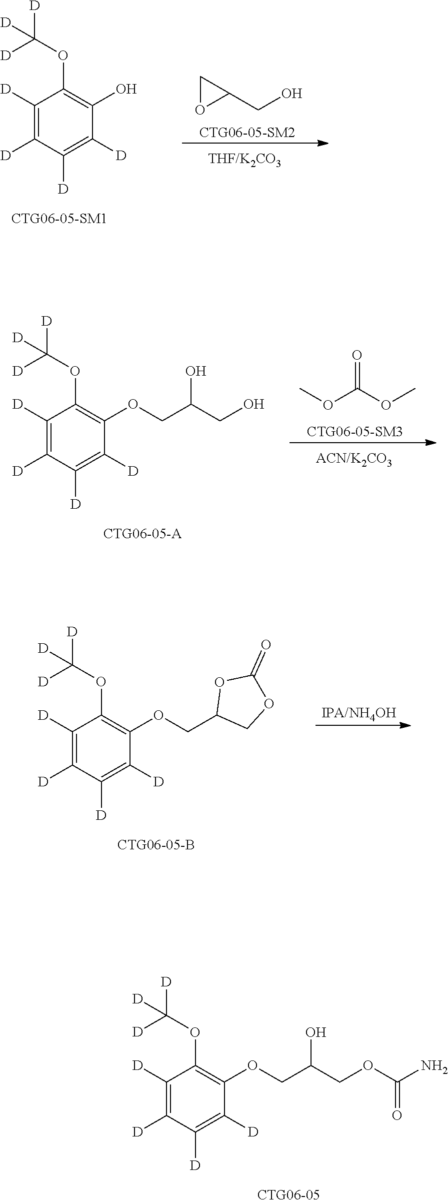

### Preparation of Intermediate CTG06-05-A

Mix CTG06-05-SM1 (175 g, 1.334 mol), CTG06-05-SM2 (108.7 g, 1.467 mol), and THF (784.0 g) in a reaction bottle and stir for 30 minutes. Add K₂CO₃ (201.8 g, 1.467 mol) to the mixture under nitrogen. Increase the temperature to 55-60° C. and stir for 16 hours. Stop the reaction after HPLC monitoring. Subsequently, lower the temperature to 0-10° C. and titrate purified water (525.0 g). Add DCM (2318.0 g) and stir for 30 minutes. Transfer the organic layer to a clean bottle and further extract the aqueous layer twice with two portions of DCM (1159.0 g each). Combine the organic layer and dry over Na₂SO₄, and then filter. Rinse the residue twice with DCM (232.0 g each). Concentrate the organic layer under vacuum using a rotary evaporator (0.08-0.10 MPa) until no fraction remains. Add acetone (1060.5 g) to the resulting solid (268.5 g). Increase the temperature to 50-60° C. and stir for 30 minutes. Turn off the heater and let the temperature drop to 15-20° C. naturally and stir for 16 hours. Continue to lower the temperature to 0-10° C., stir for 2 hours, and then filter. Vacuum-dry the solid at 50±5° C. (0.08˜0.10 MPa) for 15 hours, resulting in a pure solid of CTG06-05-A (227.5), with a yield of 83.1%.

¹HNMR (400 MHz, DMSO-d6) δ 4.90 (d, J=4.8 Hz, 1H), 4.64 (t, J=5.6 Hz, 1H), 3.93-3.97 (m, 1H), 3.77-3.86 (m, 2H), 3.43-3.47 (m, 2H).

### Preparation of Intermediate CTG06-05-B

Mix CTG06-05-A (312.5 g, 1.351 mol), CTG06-05-SM3 (486.8 g, 5.404 mol), K₂CO₃ (9.4 g, 0.068 mol), and acetonitrile (491 g) in a reaction bottle under nitrogen. Increase the temperature to 75-80° C. and stir for 18 hours. After HPLC monitoring, decrease the temperature to 0-10° C. and add purified water (1562.5 g) and DCM (4140.6 g). Stir for 30 minutes. Transfer the organic layer to a clean bottle and add purified water (1562.5 g). After 30 minutes of stirring, remove the organic layer and dry over Na₂SO₄ and then filter. Wash the residue twice with DCM (414.6 g each) and vacuum-evaporate using a rotary evaporator (0.08-0.10 MPa). Mix the resulting solid (332.0 g) and MTBE (1245.2 g) in a reaction oven and stir for 16 hours at 15-20° C. Decrease the temperature to 0-10° C. and continue to stir for 2 hours before filtration. Vacuum-dry the solid at 50±5° C. (0.08˜0.10 MPa) for 15 hours, resulting in a pure solid of CTG06-05-B (236.5 g), with a yield of 67.1‰

¹H NMR (400 MHz, DMSO-d6) δ 5.10-5.16 (m, 1H), 4.62 (t, J=8.4 Hz, 1H), 4.38-4.42 (m, 1H), 4.21 (m, 2H).

### Preparation of CTG06-05

Mix CTG06-05-B (343.5 g, 1.485 mol) and isopropyl alcohol (810.0 g) in a reaction bottle under nitrogen. Reduce the temperature to 0-10° C. and add ammonia (1875.5 g). Stir for 16 hours at room temperature. After HPLC monitoring, drop the temperature to 0-10° C. Add purified water (1030.5 g) and two portions of DCM (4551.3 g each time) for liquid-liquid extraction. Combine the organic layers and dry over Na₂SO₄ and then filter. Rinse the residue twice with DCM (455.1 g each time). Concentrate the organic layer under vacuum using a rotary evaporator (0.08-0.10 MPa) until no fraction remains. Mix the residue (370 g) and anhydrous methanol (1461.5 g) in a reaction oven. Increase the temperature to 40-45° C. to obtain a clean solution and then drop the temperature to 20-25° C. naturally. Stir for 5 hours and then filter. After rinsing twice with anhydrous methanol (146.2 g each), vacuum-dry the solid at 50±5° C. (0.08-0.10 MPa) for 15 hours, resulting in a white solid of CTG06-05 (158 g), with a yield of 42.7%.

¹H NMR (400 MHz, DMSO-d6) δ 6.51 (br, 2H), 5.20 (s, 1H), 4.03-3.86 (m, 5H).

### REFERENCES

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure. The following is the list of references:
1. LiverTox: Clinical and Research Information on Drug-Induced Liver Injury,” in Methocarbamol, Bethesda (MD), Diseases, National Institute of Diabetes and Digestive and Kidney, 2017.
2. Elenbass. J K, “Centrally acting oral skeletal muscle relaxants,” Am J Hosp Pharm, vol. 37, no. 10, pp. 1313-23, 1980.
3. Richards B L, Whittle S L, van der Heijde D M, Buchbinder R, “The efficacy and safety of muscle relaxants in inflammatory arthritis: a Cochrane systematic review,” J Rheumatol Suppl, vol. 90, pp. 34-9, 2012.
4. Beebe F A, Barkin R L, Barkin S, “A clinical and pharmacologic review of skeletal muscle relaxants for musculoskeletal conditions.” Am J Ther, vol. 12, no. 2, pp. 151-71, 2005.
5. Patanwala A E, Aljuhani O, Kopp, B, Erstad, B, “Methocarbamol use is associated with decreased hospital length of stay in trauma patients with closed rib fractures.” Am J Surg., vol. 214, no. 4, pp. 738-742, 2017.
6. Looke T D, Kluth C T, “Effect of preoperative intravenous methocarbamol and intravenous acetaminophen on opioid use after primary total hip and knee replacement.” Orthopedics, vol. 36, pp. 25-32, 2013.
7. Abd-Elsalam S, Arafa M, Elkadeem M, Elfert A, Soliman S, Elkhalawany W, Badawi R, “Randomized-controlled trial of methocarbamol as a novel treatment for muscle cramps in cirrhotic patients.” Eur J Gastroenterol Hepatol, vol. 31, no. 4, pp. 499-502, 2019.
8. Foster N E, Anema J R, Cherkin D, Chou, R, Cohen S P, Gross D P, Ferreira P H, Fritz J M, Koes B W, Peul W, Turner J A, Maher C G, “Prevention and treatment of low back pain: evidence, challenges, and promising directions.” Lancet, vol. 391, no. 10137, pp. 2368-2383, 2018.
9. Truitt E B, Patterson, R B, “Comparative hemolytic activity of mephenesin, guaiacol glycerol ether and methocarbamol in vitro and in vivo.” Proc Soc Exp Biol Med., vol. 95, no. 3, pp. 422-424, 1957.
10. O'Doherty D S, “Methocarbamol; new agent in treatment of neurological and neuromuscular diseases,” J Am Med Assoc., vol. 167, no. 2, pp. 160-163, 1958.
11. Chou R, Peterson K, Helfand M, “Comparative efficacy and safety of skeletal muscle relaxants for spasticity and musculoskeletal conditions: a systematic review,” J Pain Symptom Manage, vol. 28, no. 2, pp. 140-175, 2004.

What is claimed is:

1. A compound of the following Formula (I) or a pharmaceutically acceptable salt or ester thereof: 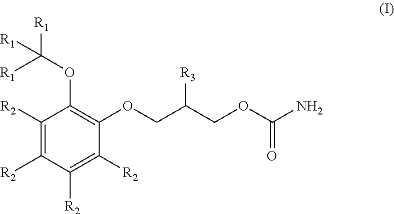
wherein each of R¹ and R² is independently selected from H, or D, and R³ is independently selected from OH, or F.

2. The compound of claim 1, wherein each of R¹ in the compound is D, each of R² is H, and R³ is OH, represented by the following structural Formula (II):

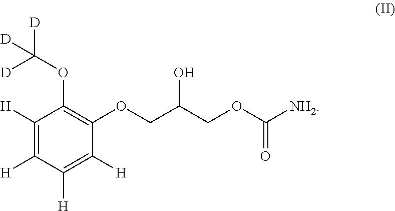 [SMILES: [2H]C([2H])([2H])Oc1ccccc1OCC(O)COC(N)=O]

3. The compound of claim 1, wherein each of R¹ in the compound is F, each of R² is H, and R³ is OH, represented by the following structural Formula (III):

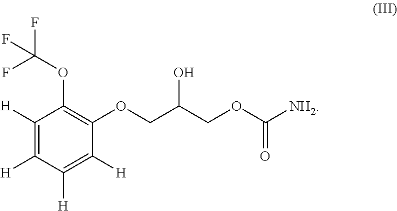 [SMILES: NC(=O)OCC(O)COc1ccccc1OC(F)(F)F]

4. The compound of claim 1, wherein each of R¹ and R² is D, and R³ is OH, represented by the following structural formula (IV):

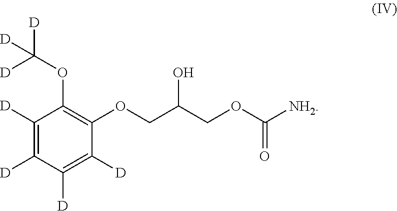 [SMILES: [2H]c1c([2H])c([2H])c(OC([2H])([2H])[2H])c(OCC(O)COC(N)=O)c1[2H]]

5. The compound of claim 1, wherein each of R¹ and R² is H, and R³ is F, represented by the following structural formula (V):

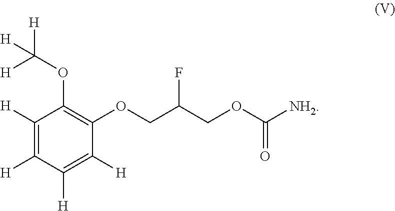 [SMILES: COc1ccccc1OCC(F)COC(N)=O]

6. A pharmaceutical composition comprising the compound of claim 1, or a pharmaceutically acceptable salt or ester thereof, useful for treating at least one muscle spasm related disease selected from the group consisting of acute pain of musculoskeletal origin, low-back pain, inflammatory arthritis, fibromyalgia, perioperative care of hip and knee replacements, and related diseases and conditions.

7. A unit dosage form comprising the pharmaceutical composition of claim 1, wherein the dosage form is suitable for administrating to a subject, animal or human, that suffers from at least one muscle-spasm related disease selected from the group consisting of acute pain of musculoskeletal origin, low-back pain, inflammatory arthritis, fibromyalgia, perioperative care of hip and knee replacements, and related diseases and conditions.

8. A synthetic method of producing the compound of claim 1.

9. A pharmaceutical composition comprising the compound of claim 2, or a pharmaceutically acceptable salt or ester thereof, useful for treating at least one muscle spasm related disease selected from the group consisting of acute pain of musculoskeletal origin, low-back pain, inflammatory arthritis, fibromyalgia, perioperative care of hip and knee replacements, and related diseases and conditions.

10. A unit dosage form comprising the pharmaceutical composition of claim 2, wherein the dosage form is suitable for administrating to a subject, animal or human, that suffers from at least one muscle-spasm related disease selected from the group consisting of acute pain of musculoskeletal origin, low-back pain, inflammatory arthritis, fibromyalgia, perioperative care of hip and knee replacements, and related diseases and conditions.

11. A pharmaceutical composition comprising the compound of claim 3, or a pharmaceutically acceptable salt or ester thereof, useful for treating at least one muscle spasm related disease selected from the group consisting of acute pain of musculoskeletal origin, low-back pain, inflammatory arthritis, fibromyalgia, perioperative care of hip and knee replacements, and related diseases and conditions.

12. A unit dosage form comprising the pharmaceutical composition of claim 3, wherein the dosage form is suitable for administrating to a subject, animal or human, that suffers from at least one muscle-spasm related disease selected from the group consisting of acute pain of musculoskeletal origin, low-back pain, inflammatory arthritis, fibromyalgia, perioperative care of hip and knee replacements, and related diseases and conditions.

13. A pharmaceutical composition comprising the compound of claim 4, or a pharmaceutically acceptable salt or ester thereof, useful for treating at least one muscle spasm related disease selected from the group consisting of acute pain of musculoskeletal origin, low-back pain, inflammatory arthritis, fibromyalgia, perioperative care of hip and knee replacements, and related diseases and conditions.

14. A unit dosage form comprising the pharmaceutical composition of claim 4, wherein the dosage form is suitable for administrating to a subject, animal or human, that suffers from at least one muscle-spasm related disease selected from the group consisting of acute pain of musculoskeletal origin, low-back pain, inflammatory arthritis, fibromyalgia, perioperative care of hip and knee replacements, and related diseases and conditions.

15. A pharmaceutical composition comprising the compound of claim 5, or a pharmaceutically acceptable salt or ester thereof, useful for treating at least one muscle spasm related disease selected from the group consisting of acute pain of musculoskeletal origin, low-back pain, inflammatory arthritis, fibromyalgia, perioperative care of hip and knee replacements, and related diseases and conditions.

16. A unit dosage form comprising the pharmaceutical composition of claim 5, wherein the dosage form is suitable for administrating to a subject, animal or human, that suffers from at least one muscle-spasm related disease selected from the group consisting of acute pain of musculoskeletal origin, low-back pain, inflammatory arthritis, fibromyalgia, perioperative care of hip and knee replacements, and related diseases and conditions.
